(19) United States
(12) Patent Application Publication
(10) Pub. No.: US 2011/0129826 A1
(43) Pub. Date: Jun. 2, 2011

(54) METHOD FOR DETERMINATION OF INFLAMMATORY DISEASE BY USING SINGLE NUCLEOTIDE POLYMORPHISM IN BRCA1-RELATED PROTEIN (BRAP) GENE

(75) Inventors: Toshihiro Tanaka, Tokyo (JP); Kouichi Ozaki, Tokyo (JP)

(73) Assignee: RIKEN, Saitama (JP)

(21) Appl. No.: 12/739,494

(22) PCT Filed: Oct. 29, 2008
(86) PCT No.: PCT/JP2008/069654

§ 371 Date: Nov. 2, 2010

(30) Foreign Application Priority Data
Oct. 29, 2007 (JP) 2007-279850

(51) Int. Cl. C12Q 1/68; C07H 21/04

(52) U.S. Cl. 435/6.11; 536/24.31; 536/24.33

(57) ABSTRACT

It is an object of the present invention to identify a novel single nucleotide polymorphism (SNP) associated with the development and advancement of inflammatory diseases such as myocardial infarction. The present invention provides a method for judging inflammatory diseases, which comprises detecting at least one gene polymorphism in the BRCA1-associated protein (BRAP) gene.

### TECHNICAL FIELD

The present invention relates to a method for judging inflammatory diseases comprising detecting the gene polymorphisms in the BRCA1-associated protein (BRAP) gene, oligonucleotides used for said method, a diagnostic kit for inflammatory diseases comprising said oligonucleotides, and uses thereof.

### BACKGROUND ART

In spite of changes in lifestyle and novel pharmacological approaches, coronary artery diseases including myocardial infarction are the leading causes of death in the world. Accordingly, identification of genetic and environmental factors associated with the development of such diseases has been strongly desired.

A common genetic variation is known to be deeply associated with the risk of affliction with lifestyle-related diseases such as diabetes or hypertension. Susceptibility genes for polygenic diseases are identified by a method that involves the use of “genetic linkage” and by a method that involves the use of “association.” Via analysis of genetic linkage, whether or not the locus of the disease susceptibility gene is linked to the locus of the gene marker (mainly microsatellites) is detected, i.e., the relationship between the loci is inspected. In the association analysis, the type (allele) of gene marker (mainly single nucleotide polymorphisms, i.e., SNPs) associated with a disease is detected, i.e., the relationship between alleles is inspected via association analysis. Accordingly, it can be said that association analysis, which involves the use of a common variation as a marker, is more reliable than genetic linkage analysis, which involves the inspection of the localisation of disease-associated genes. Single nucleotide polymorphisms (SNPs) are useful polymorphism markers when searching for genes associated with the incidence of a disease or drug reactivity. SNPs may directly influence the quality or quantity of gene products or may increase the risk of serious side effects resulting from given diseases or drugs. Thus, search of a larger number of SNPs may contribute to the identification of disease-associated genes or the establishment of a diagnostic method that prevents side effects that result from the use of drugs.

The correlation between genetic variation and myocardial infarction has been evaluated by, for example, a method for analyzing the polymorphisms of a lymphotoxin-α (LT-α) gene, an I Kappa B-like (IKBL) gene, or a BAT1-gene to determine the genetic factors of myocardial infarction (International Publication WO2004/015100), a method for analyzing the polymorphisms of a galectin-2 gene to determine the genetic factors of myocardial infarction (International Publication WO2005/017200), and the like. In addition, the correlation between SNP in an aldehyde dehydrogenase 2 gene and myocardial infarction has also been reported (S. Takagi et al., Hypertens Res. Vol. 25, No. 5 (2002), pages 677-681). However, according to this report, both the number of samples and the P value have not been sufficient, and thus the reliability of the data has been low. That is to say, since only the SNP of ALDH2 has been analyzed using a small number of samples in this literature, the data may include genotyping errors. As a result, the reliability of the obtained results has been low, and the significance thereof has also been low. As described above, the genetic variation associated with myocardial infarction has not yet been fully elucidated.
Non-Patent Document 1: S. Takagi et al., Hypertens Res. Vol. 25, No. 5 (2002), pages 677-681
Patent Document 1: International Publication WO2004/015100
Patent Document 2: International Publication WO2005/017200

### DISCLOSURE OF THE INVENTION

### Object to be Solved by the Invention

It is an object of the present invention to identify a novel single nucleotide polymorphism (SNP) associated with the development and advancement of inflammatory diseases such as myocardial infarction. It is another object of the present invention to provide a method for diagnosing inflammatory diseases such as myocardial infarction or a method for developing a therapeutic agent for such inflammatory diseases, utilizing the identified SNP.

### Means for Solving the Object

The present inventors have conducted intensive studies directed towards achieving the aforementioned objects. As a result, the inventors have discovered that a novel single nucleotide polymorphism (SNP) in a BRCA1-associated protein (BRAP) gene is associated with the development and advancement of myocardial infarction, thereby completing the present invention.

(1) A method for judging inflammatory diseases, which comprises detecting at least one gene polymorphism in the BRCA1-associated protein (BRAP) gene.
(2) A method for judging inflammatory diseases, which comprises detecting at least one single nucleotide polymorphism in the BRCA1-associated protein (BRAP) gene.
(3) A method for judging inflammatory diseases, which comprising detecting any one of the following polymorphisms:
(i) the A/G polymorphism at nucleotide 90 in the nucleotide sequence of exon 5 of the BRCA1-associated protein (BRAP) gene as shown in SEQ ID NO: 1 (registration No. rs3782886 in the NCBI SNP Database);
(ii) the A/G polymorphism at nucleotide 270 in the nucleotide sequence of intron 3 of the BRCA1-associated protein (BRAP) gene as shown in SEQ ID NO: 2 (registration No. rs110660001 in the NCBI SNP Database); and
(iii) a polymorphism that is in a linkage disequilibrium state in which an r-square value used as a linkage disequilibrium index is 0.8 or greater (preferably, 0.9 or greater) with respect to the polymorphism described in (i) or (ii) above.
(4) The method according to any one of (1) to (3), wherein the inflammatory disease is myocardial infarction.
(5) An oligonucleotide, which can hybridize to a sequence consisting of at least 10 continuous nucleotides including the A/G polymorphism at nucleotide 90 in the nucleotide sequence of exon 5 of the BRCA1-associated protein (BRAP) gene as shown in SEQ ID NO: 1 or at nucleotide 270 in the nucleotide sequence of intron 3 of the BRCA1-associated protein (BRAP) gene as shown in SEQ ID NO: 2, or a complementary sequence thereof, and which is used as a probe in any one of the methods according to (1) to (4).
(6) An oligonucleotide, which can amplify a sequence consisting of at least 10 continuous nucleotides including the A/G polymorphism at nucleotide 90 in the nucleotide sequence of exon 5 of the BRCA1-associated protein (BRAP) gene as shown in SEQ ID NO: 1 or at nucleotide 270 in the nucleotide sequence of intron 3 of the BRCA1-associated protein (BRAP) gene as shown in SEQ ID NO: 2, and/or a complementary sequence thereof, and which is used as a primer in any one of the methods according to (1) to (4).
(7) The oligonucleotide according to (6), wherein the primer is a forward primer and/or a reverse primer.
(8) A kit for diagnosing inflammatory diseases, which comprises at least one oligonucleotide according to any one of (5) to (7).
(9) The kit according to (8), wherein the inflammatory disease is myocardial infarction.
(10) A method for analyzing the expression state of a BRCA1-associated protein (BRAP), which comprises detecting the A/G polymorphism at nucleotide 90 in the nucleotide sequence of exon 5 of the BRCA1-associated protein (BRAP) gene as shown in SEQ ID NO: 1 or the A/G polymorphism at nucleotide 270 in the nucleotide sequence of intron 3 of the BRCA1-associated protein (BRAP) gene as shown in SEQ. ID NO: 2.
(11) A method for screening for a therapeutic agent for inflammatory diseases, which comprises steps of analyzing the expression level of a BRCA1-associated protein (BRAP) or the function of a BRCA1-associated protein (BRAP) in cells in the presence of a candidate substance, and selecting a substance that suppresses said expression level or a substance that inhibits or modifies said function.
(12) A method for measuring the transcriptional activity of a BRCA1-associated protein (BRAP), which comprises introducing a BRAP gene fragment containing the A/G polymorphism at nucleotide 270 in the nucleotide sequence of intron 3 of the BRCA1-associated protein (BRAP) gene as shown in SEQ ID NO: 2 into cells, culturing the cells, and analyzing the expression of said gene.
(13) A method for screening for a substance that inhibits or promotes the transcriptional activity of a BRCA1-associated protein (BRAP), which comprises introducing a BRAP gene fragment containing the A/G polymorphism at nucleotide 270 in the nucleotide sequence of intron 3 of the BRCA1-associated protein (BRAP) gene as shown in SEQ ID NO: 2 into cells, culturing the cells in the presence of a candidate substance that inhibits or promotes the transcriptional activity of BRAP, and analyzing the expression of said gene.
(14) A method for screening for a transcription-regulatory factor of a BRCA1-associated protein (BRAP), which comprises bringing a BRAP gene fragment containing the A/G polymorphism at nucleotide 270 in the nucleotide sequence of intron 3 of the BRCA1-associated protein (BRAP) gene as shown in SEQ ID NO: 2 into contact with a sample assumed to comprise the transcription-regulatory factor of BRAP, and detecting the binding between the aforementioned gene fragment and the transcription-regulatory factor.

### Effect of the Invention

According to the present invention, a novel single nucleotide polymorphism (SNP) associated with the development and advancement of inflammatory diseases such as myocardial infarction has been identified. Utilizing the SNP identified by the present invention, it becomes possible to provide a method for diagnosing inflammatory diseases such as myocardial infarction, or a method for developing a therapeutic agent for such inflammatory diseases.

### BEST MODE FOR CARRYING OUT THE INVENTION

In the present invention, it has been found that a BRCA1-associated protein (BRAP) gene product binds to a galectin-2 gene product which is known as a gene product susceptible to inflammatory diseases such as myocardial infarction. Utilizing SNPs in the BRCA1-associated protein (BRAP) gene which was identified by the present invention, it becomes possible to develop novel diagnostic and preventive methods for inflammatory diseases such as myocardial infarction and therapeutic agents therefor. Hereafter, the embodiments of the present invention will be described in greater detail.

### [1] Method for Judging Inflammatory Diseases

The method of the present invention comprises detecting the gene polymorphisms (particularly, single nucleotide polymorphisms (SNPs)) in the BRCA1-associated protein (BRAP) gene associated with inflammatory diseases, so as to judging the presence or absence of the onset of inflammatory diseases or so as to evaluate the possibility of the onset of inflammatory diseases.

In the present invention, the description “detecting at least one gene polymorphism (e.g., a single nucleotide polymorphism) in the BRCA1-associated protein (BRAP) gene” refers to: (i) direct detection of the gene polymorphism (referred to as “a polymorphism in the gene”); and (ii) detection of a gene polymorphism in the complementary sequence of the aforementioned gene (referred to as “a polymorphism in the complementary sequence”), and deduction of the existence of the polymorphism in the gene based on the results of such detection. Since the nucleotides of the gene are not always completely complementary to the nucleotides of the complementary sequence, it is preferable to directly detect the polymorphisms in the gene.

Specific examples of a preferred gene polymorphism in the BRCA1-associated protein (BRAP) gene include:

(i) the A/G polymorphism at nucleotide 90 in the nucleotide sequence of exon 5 of the BRCA1-associated protein (BRAP) gene as shown in SEQ ID NO: 1 (registration No. rs3782886 in the NCBI SNP Database);
(ii) the A/G polymorphism at nucleotide 270 in the nucleotide sequence of intron 3 of the BRCA1-associated protein (BRAP) gene as shown in SEQ ID NO: 2 (registration No. rs110660001 in the NCBI SNP Database); and
(iii) a polymorphism that is in a linkage disequilibrium state in which an r-square value used as a linkage disequilibrium index is 0.8 or greater (preferably, 0.9 or greater) with respect to the polymorphism described in (i) or (ii) above.

It is to be noted that “r” at nucleotide 90 as shown in SEQ ID NO: 1 indicates either A or G In addition, “r” at nucleotide 270 as shown in SEQ ID NO: 2 indicates either A or G

The above-mentioned polymorphism that is in a linkage disequilibrium state in which an r-square value used as a linkage disequilibrium index is 0.8 or greater (preferably, 0.9 or greater) with respect to the polymorphism described in (i) or (ii) above may be a polymorphism in the BRCA1-associated protein (BRAP) gene or a polymorphism in an FLJ30092 gene. Alternatively, it may also be a polymorphism in genes other than the above-mentioned two types of genes. It is to be noted that the FLJ30092 gene indicates an AF-1-specific protein phosphatase gene.

Specific examples of such polymorphism that is in a linkage disequilibrium state in which an r-square value used as a linkage disequilibrium index is 0.8 or greater (preferably, 0.9 or greater) with respect to the polymorphism described in (i) or (ii) above include an SNP in the FLJ30092 gene (registration No. rs2074356 in the NCBI SNP Database) and two SNPs in an aldehyde dehydrogenase 2 family (ALDH2) gene (registration Nos. rs671 and rs4646776 in the NCBI SNP Database).

As shown in Table 4 later, for example, when nucleotide 90 in the nucleotide sequence of exon 5 of the BRCA1-associated protein (BRAP) gene as shown in SEQ ID NO: 1 is A, it can be determined that inflammatory disease has not been developed, or that the possibility of developing this disease is low.

In contrast, when nucleotide 90 in the nucleotide sequence of exon 5 of the BRCA1-associated protein (BRAP) gene as shown in SEQ ID NO: 1 is G, it can be determined that inflammatory disease has been developed, or that the possibility of developing this disease is high.

In the present invention, it is also possible to use a polymorphism that is in a linkage disequilibrium state in which a linkage disequilibrium index Δ² is 0.8 or greater (preferably, 0.9 or greater) with respect to the polymorphism described in (i) or (ii) above.

It is assumed that there are two linkage loci, for example. In each locus, there are two alleles (biallelic loci, like SNP). The alleles in the first locus are defined as 1 and 2, and the alleles in the second locus are defined 1 and 2 (needless to say, the allele 1 in the first focus is different from the allele 1 in the second locus). If there is not a linkage disequilibrium between the first and second loci, there are four haplotypes depending on whether the chromosome has the allele 1 or 2 of the first locus and the allele 1 or 2 of the second locus. The frequency of each haplotype becomes as follows.

TABLE 1
Haplotype frequency

| Haplotype (first locus-second locus) | Frequency |
|---|---|
| 1-1 | p11 |
| 1-2 | p12 |
| 2-1 | p21 |
| 2-2 | p22 |

In this case, the frequency of the alleles 1 and 2 in each locus becomes as follows.

TABLE 2
Allele frequency

| Allele | Frequency |
|---|---|
| Allele 1 in first locus | p1 · = p11 + p12 |
| Allele 2 in first locus | p2 · = p21 + p22 = 1 − p1 · |
| Allele 1 in second locus | p · 1 = p11 + p21 |
| Allele 2 in second locus | p · 2 = p12 + p22 = 1 − p · 1 |

The symbol “·” is not a symbol as a product, but is a dot.
The symbol “P1 ·” indicates a variable.

If there is not a linkage disequilibrium, the frequency of the haplotype 1-1 is the product of the frequencies of the alleles 1 of the first and second loci. That is to say, if there is not a linkage disequilibrium, p11=p1·p·1.

However, if there is a linkage disequilibrium, the above formula does not hold. If such deviation level is expressed with D as in the following formula, D=p11−p1·p·1. Thus, the frequencies of the four haplotypes can be expressed as follows, using the frequencies of the alleles and D.

TABLE 3
Haplotype frequency in presence of linkage disequilibrium

| Allele | Frequency |
|---|---|
| 1-1 | p11 = p1 · p · 1 + D |
| 1-2 | p12 = p1 · p · 2 − D |
| 2-1 | p21 = p2 · p · 1 − D |
| 2-2 | p22 = p2 · p · 2 + D |

The “D” indicates a linkage disequilibrium coefficient. Such D can also be expressed as follows:

D=p11p22−p12p21.

When there is not a linkage disequilibrium, D=0. When D>0, it is referred to as a positive linkage disequilibrium.

If “r” indicates a recombination rate, D decreases at a rate of r per generation. That is, if the linkage disequilibrium coefficient of a certain generation is defined as D, the linkage disequilibrium coefficient Dₙ after an n number of generations is

Dₙ=(1−r)ⁿD.

As a matter of fact, since all of haplotype frequencies and allele frequencies must have values between 0 and 1, the range of D is limited as follows.

That is, when D>0 or D=0, the maximum value that D may have is

Dₘₐₓ=min(p1·p·2,p2·p·1).

When D<0, the minimum value that D may have is

Dₘᵢₙ=max(−p1·p·1,−p2·p·2).

Accordingly, the linkage disequilibrium coefficient may also be expressed with the following values obtained by standardizing the D:

D′=D/Dₘₐₓ (when D is a positive value); and

D′=D/Dₘᵢₙ (when D is a negative value) [however, when D<0, some study papers define D′=D/Dₘᵢₙ].

According to the above-described definitions, D′ always becomes a positive value or 0 (zero). [However, if the definition in the parentheses [ ] above is adopted, D<0.]

Other than such D or D′, the following p may also be used. The symbol ρ² is equal to χ²/n, and it is often expressed as Δ².

Δ²=ρ²=D²/(p1·p2·p·1p·2)

The commonly used indexes of linkage disequilibrium, including those as described above, are shown below.

$$D = {p}_{11} {p}_{22} - {p}_{12} {p}_{21}$$

$$Δ = \frac{{p}_{11} {p}_{22} - {p}_{12} {p}_{21}}{{( {p}_{1} \cdot {p}_{2} \cdot p {\cdot }_{1} p {\cdot }_{2} )}^{1 / 2}}$$

$${Δ}^{2} = \frac{{( {p}_{11} {p}_{22} - {p}_{12} {p}_{21} )}^{2}}{{p}_{1} \cdot {p}_{2} \cdot p {\cdot }_{1} p {\cdot }_{2}}$$

$${D}^{′} = \{ \begin{matrix}\frac{{p}_{11} {p}_{22} - {p}_{12} {p}_{21}}{min ( {p}_{1} \cdot p {\cdot }_{2} , p {\cdot }_{1} {p}_{2} \cdot )} & D > O \\ \frac{{p}_{11} {p}_{22} - {p}_{12} {p}_{21}}{min ( {p}_{1} \cdot p {\cdot }_{1} , p {\cdot }_{2} {p}_{2} \cdot )} & D < O\end{matrix} \\ δ = \frac{{p}_{11} {p}_{22} - {p}_{12} {p}_{21}}{p {\cdot }_{1} {p}_{22}} \\ d = \frac{{p}_{11} {p}_{22} - {p}_{12} {p}_{21}}{p {\cdot }_{1} p {\cdot }_{2}} \\ Q = \frac{{p}_{11} {p}_{22} - {p}_{12} {p}_{21}}{{p}_{11} {p}_{22} \cdot {p}_{12} {p}_{21}}$$

The above-described Δ² (or ρ²) has the same definitions as those of a linkage disequilibrium index, r-square.

Among the above-described polymorphisms, those, in which one allele frequency is significantly higher in any given affected patient population than in any given unaffected patient population, can be used for the diagnostic method of the present invention.

The affected patient population and unaffected patient population are not limited, in terms of the size (the number of samples), the background of each sample (e.g. hometown, age, sex, disease, etc.) and the like, as long as these populations are constituted with a sufficient number of samples that are enough to give statistically reliable results.

In the present description, the term “judging” of diseases is used to mean the judgment of the development of diseases, the judgment of a possibility of affliction with diseases (a prediction of morbidity), elucidation of genetic factors of diseases, and the like.

The “judgment” of diseases can be carried out based on the results of the aforementioned method for detecting single nucleotide polymorphisms in combination with another form of polymorphism analysis (VNTR or RFLP) and/or the results of other forms of detection, according to need.

In the present description, the term “inflammatory diseases” is not particularly limited, as long as induction of cell-adhesion factors or cytokines, which are known to be correlated with the inflammatory conditions, is observed. Examples of such diseases include chronic rheumatism, systemic lupus erythematodes, inflammatory enteritis, various allergy reactions, bacterial shock, and arteriosclerotic diseases such as myocardial infarction or cerebral apoplexy. A specific example thereof is myocardial infarction.

### (Target of Detection)

The target of gene polymorphism detection is preferably genomic DNA. According to the circumstances (i.e., when the sequences of a polymorphism site and the region in the vicinity thereof are identical or completely complementary to the genome), cDNA or mRNA can be used. Such target can be obtained from samples, such as any biological samples: for example, body fluid such as blood, bone marrow fluid, semen, peritoneal fluid, or urine; tissue cells from the liver and the like; and body hair such as hair. Genomic DNA and the like can be extracted from such samples and purified in accordance with a conventional technique.

### (Amplification)

In order to detect a gene polymorphism, a region containing a gene polymorphism is first amplified. Amplification is carried out via, for example, PCR. It can also be carried out via other conventional amplification techniques, such as the NASBA method, the LCR method, the SDA method, or the LAMP method.

Primers are selected so as to be capable of amplifying, for example, a sequence consisting of at least 10 continuous nucleotides, preferably 10 to 100 nucleotides, and more preferably 10 to 50 nucleotides, containing the aforementioned single nucleotide polymorphism site in the sequence as shown in SEQ ID NO: 1 or SEQ ID NO: 2, and/or a complementary sequence thereof.

Primers may contain one or more substitutions, deletions, or additions in such sequence, as long as they can function as primers for amplifying a sequence consisting of a given number of nucleotides including the aforementioned single nucleotide polymorphism site.

One of the forward or reverse primers, which hybridize to the single nucleotide polymorphism site so that amplification is carried out only when the sample has a single allele, may be selected as the primer for amplification. Primers can be labeled with a fluorescent or radioactive substance, according to need.

### (Detection of Gene Polymorphism)

The gene polymorphism can be detected via hybridization with a probe specific to an allele. Probes may be labeled with an adequate means such as a fluorescent or radioactive substance, according to need. Probes are not particularly limited, as long as they contain the aforementioned single nucleotide polymorphism site, hybridize to the test material, and impart detectable specificity under the detection conditions to be employed. An oligonucleotide that can hybridize to a sequence consisting of at least 10 continuous nucleotides, preferably 10 to 100 nucleotides, and more preferably 10 to 50 nucleotides, including the aforementioned single nucleotide polymorphism site in the sequence as shown in SEQ ID NO: 1 or SEQ ID NO: 2, or a complementary sequence thereof, can be employed as a probe. An oligonucleotide is preferably selected in such a way that the single nucleotide polymorphism is located in substantially the center of the probe. Such oligonucleotide may comprise one or more substitutions, deletions, or additions in such sequence, as long as it can function as a probe, i.e., it hybridizes to the sequence having the target allele but it does not hybridize to a sequence having another allele. Also, a probe may satisfy the aforementioned requirement by annealing to the genomic DNA to form a cyclic structure, as with the case of a single-strand probe (padlock probe) used for RCA (rolling circle amplification) amplification.

The hybridization conditions employed in the present invention are those sufficient for distinguishing alleles. An example of such conditions is stringent conditions where hybridization takes place when a sample has a single allele and does not take place when a sample has another allele. Examples of “stringent conditions” include those described in the Molecular Cloning: A Laboratory Manual, vol. 2 (Sambrook et al., 1989). Under stringent conditions, for example, hybridization takes place in a solution containing 6×SSC (the composition of 1×SSC: 0.15M NaCl, 0.015M sodium citrate, pH 7.0), 0.5% SDS, 5×Denhart's solution, and 100 mg/ml of herring sperm DNA while being incubated with a probe at 65° C. overnight.

A probe can also be used as a DNA chip by immobilizing one end thereof on a substrate. In such a case, a DNA chip may have immobilized thereon only a probe corresponding to a single allele or a probe corresponding to both alleles.

The gene polymorphism can also be detected via analysis of restriction fragment length polymorphism (RFLP). According to this technique, the sample nucleic acid, where whether or not it should be cleaved with a restriction enzyme depends on the genotype at the SNP site, is digested with a restriction enzyme, and the size of the digested fragment is inspected to determine whether or not the sample nucleic acid was cleaved with the restriction enzyme. Thus, the polymorphisms of the sample are analyzed.

The gene polymorphisms may be detected by directly sequencing the amplification product (a method of direct sequencing). Sequencing can be carried out via conventional techniques, such as the dideoxy method or the Maxam-Gilbert method.

The gene polymorphisms can be detected via, for example, denaturing gradient gel electrophoresis (DGGE), single strand conformation polymorphism (SSCP), allele-specific PCR, hybridization utilizing allele-specific oligonucleotides (ASO), chemical cleavage of mismatches (CCM), the heteroduplex method (HET), primer extension (PEX), or rolling circle amplification (RCA).

### [2] Kit for Diagnosing Inflammatory Diseases

The aforementioned oligonucleotide used as a primer or a probe can be provided as a kit for diagnosing inflammatory diseases comprising such oligonucleotide. This kit may comprise a restriction enzyme, polymerase, nucleoside triphosphate, a label, a buffer, and the like that are used for the method for analyzing gene polymorphisms.

### [3] Method for Analyzing Expression State of BRCA1-Associated Protein (BRAP)

According to the present invention, the expression state of a BRCA1-associated protein (BRAP) can be analyzed by detecting the above-described single nucleotide polymorphisms.

When the nucleotide 270 in the nucleotide sequence of intron 3 of the BRCA1-associated protein (BRAP) gene as shown in SEQ ID NO: 2 is G (BRAP intron 3 270G), for example, the expression level of the BRAP can be determined to be high. In contrast, when nucleotide 270 in the nucleotide sequence of intron 3 of the BRCA1-associated protein (BRAP) gene as shown in SEQ ID NO: 2 is A (BRAP intron 3 270A), the expression level of the BRAP can be determined to be low.

### [4] Method for Screening for Therapeutic Agent for Inflammatory Diseases

According to the present invention, a therapeutic agent for inflammatory diseases can be screened for by analyzing the expression level of a BRCA1-associated protein (BRAP) gene in cells in the presence of a candidate substance and selecting a substance that suppresses such expression level. For example, the expression level of the BRCA1-associated protein (BRAP) gene in cells is analyzed in the presence of a candidate substance, and a substance that decreases such expression level can be selected.

For example, such screening can be carried out via a step of bringing cells into contact with a candidate substance, a step of analyzing the expression level of the BRCA1-associated protein (BRAP) gene in the cells, and a step of selecting a candidate substance, as a therapeutic agent for inflammatory diseases, that can alter the expression level of such gene, compared with the conditions that pertain in the absence of the candidate substance.

Any substance can be employed as a candidate substance. The type of candidate substance is not particularly limited, and an individual low-molecular-weight synthetic compound, a compound existing in an extract of a naturally occurring substance, a compound library, a phage display library, or a combinatorial library may be used. Preferably, a candidate substance is a low-molecular-weight compound, and a compound library of low-molecular-weight compounds is also preferable. A compound library can be constructed by a method known in the art. A commercially available compound library can also be used

### [5] Method for Measuring Transcriptional Activity of BRCA1-Associated Protein (BRAP)

According to the present invention, a BRCA1-associated protein (BRAP) gene fragment containing the aforementioned single nucleotide polymorphism is introduced into cells, the cells are then cultured, and the expression of such gene is then analyzed. Thus, the transcriptional activity of the BRCA1-associated protein (BRAP) can be measured.

According to a preferred embodiment of the present invention, a transcription unit comprising a reporter gene ligated to a site downstream of the aforementioned BRCA1-associated protein (BRAP) gene fragment is introduced into cells, such cells are then cultured, and reporter activity is then measured to analyze the expression of such gene.

When a single nucleotide polymorphism is present at the promoter site, for example, cultured cells where a system comprising a reporter gene ligated to a site downstream of the gene containing such single nucleotide polymorphism has been introduced are cultured, and the reporter activity is then measured. Thus, differences in transcription efficiency resulting from single nucleotide polymorphism can be assayed.

Examples of the reporter genes used herein include luciferase, chloramphenicol, acetyltransferase, and galactosidase genes.

[6] Method for Screening for Substance that Inhibits or Promotes Transcriptional Activity of BRCA1-Associated Protein (BRAP)

According to the present invention, a BRCA1-associated protein (BRAP) gene fragment containing the aforementioned single nucleotide polymorphism is introduced into cells, such cells are then cultured in the presence of a candidate substance that inhibits or promotes the transcriptional activity of a BRCA1-associated protein (BRAP), and the expression of such gene is then analyzed. Thus, a substance that inhibits or promotes the transcriptional activity of the BRCA1-associated protein (BRAP) can be screened.

According to a preferred embodiment of the present invention, a transcription unit comprising a reporter gene ligated to a site downstream of the aforementioned BRCA1-associated protein (BRAP) gene fragment is introduced into cells, such cells are then cultured, and the reporter activity is measured to analyze the expression of such gene.

For example, a system comprising a reporter gene inserted into a site downstream of a gene having a single nucleotide polymorphism (e.g. BRAP intron 3 270G) causing a significantly high expression level of a BRCA1-associated protein (BRAP) is introduced into cells. Thereafter, the cells are cultured in the presence of and in the absence of a candidate substance. If the reporter activity decreases when the culture is conducted in the presence of a candidate compound, this candidate compound can be selected as a substance that inhibits the transcriptional activity of the BRCA1-associated protein (BRAP).

Herein, the reporter genes as mentioned above can be employed.

Any substance can be employed as a candidate substance. The type of candidate substance is not particularly limited, and an individual low-molecular-weight synthetic compound, a compound existing in an extract of a naturally occurring substance, a compound library, a phage display library, or a combinatorial library may be used. Preferably, a candidate substance is a low-molecular-weight compound, and a compound library of low-molecular-weight compounds is also preferable. A compound library can be constructed by a method known in the art. A commercially available compound library can also be used.

The present invention also includes a substance that inhibits or promotes the transcriptional activity of a BRCA1-associated protein (BRAP) which is obtained by the aforementioned screening method of the present invention. Such substance that inhibits the transcriptional activity of the BRCA1-associated protein (BRAP) is useful as a candidate substance of various agents, such as a therapeutic agent for myocardial infarction, an anti-inflammatory agent, and an immunosuppressive agent

### [7] Method for Screening for Transcription-Regulatory Factor of BRCA1-Associated Protein (BRAP)

According to the present invention, the aforementioned gene fragment that contains a single nucleotide polymorphism is brought into contact with a sample, which is deduced to contain a transcription-regulatory factor of a BRCA1-associated protein (BRAP), and the binding between such fragment and the transcription-regulatory factor is detected. Thus, a transcription-regulatory factor of the BRCA1-associated protein (BRAP) can be screened. The binding between the gene fragment that contains a single nucleotide polymorphism and the substance, which is deduced to contain a transcription-regulatory factor of the BRCA1-associated protein (BRAP), is detected via gel-shift analysis (electrophoretic mobility shift assay (EMSA)), DNase I footprinting, or the like, with gel-shift assay being preferable. In gel-shift assay, molecular size is enlarged upon binding of proteins (transcription-regulatory factors), and this results in lowered mobility of DNA in electrophoresis. Accordingly, a ³²P-labeled gene fragment is mixed with a transcription-regulatory factor and the resultant is subjected to gel electrophoresis. When the position of DNA is observed via autoradiography, DNA to which a transcription-regulatory factor has been bound is found to move slowly. Thus, it is detected as a band that moves more slowly than usual bands.

[8] Method for Screening for Agent that Inhibits or Modifies BRCA1-Associated Protein

In the present invention, the function of a BRCA1-associated protein (BRAP) is analyzed in the presence of a candidate substance, and a substance that inhibits or modifies such function is then selected, so as to screen for an agent that inhibits or modifies the BRCA1-associated protein. The functions of BRAP include, but are not limited to a function to bind to galectin-2 and a function to maintain or improve the activity of NFκB. Accordingly, the binding state between the BRCA1-associated protein (BRAP) and the galectin-2 is analyzed in the presence or absence of a candidate substance, for example, and a substance that inhibits the aforementioned binding can be selected as an agent that inhibits the BRCA1-associated protein. Otherwise, the activity of NFκB is measured in cells, tissues, organs or individuals that express the BRCA1-associated protein (BRAP), in the presence or absence of a candidate substance, and a substance that decreases the NFκB activity can be selected as an agent that inhibits the BRCA1-associated protein.

Hereinafter, the present invention will be described more in detail in the following examples. However, these examples are not intended to limit the scope of the present invention.

### EXAMPLES

Hereinafter, the present invention will be more specifically described in the following examples.

### Example 1

### Identification of BRCA1-Associated Protein (BRAP) that is Protein Binding to Galectin-2

Using proteins extracted from HeLa cells, a novel protein that binds to a myocardial infarction susceptibility gene product, galectin-2, was screened for by tandem affinity purification.

Specifically, a tandem affinity purification operation was carried out by basically applying the method of Rigaut et al. (Rigaut, G et al., A generic protein purification method for protein complex characterization and proteome exploration. Nature Biotechnol. 17, 1030-1032 (1999)), and adding several modifications to the aforementioned method.

Using a pCMV-Myc vector (Sigma), a fusion expression cassette encoding a TEV cleavage site, an S tag and a His tag was constructed as a TAP tag sequence. The constructed TAP vector expresses a target protein having a TAP tag at the C-terminus thereof and an Myc tag at the N-terminus thereof in mammalian cells under the control of a Cytomegalovirus promoter. Using Fugene (Roche), HeLa cells (Health Science Research Resources Bank; obtained from JCRB9004) were transiently transfected with a galectin-2 TAP vector or a TAP vector as a negative control. The transfected cells were dissolved with a protein extraction reagent (Clontech) that contained a single complete protease inhibitor tablet (Roche) per 20 ml and 5 μg ml⁻¹ MG-132 (Calbiochem) on ice, and the obtained solution was then diluted by a factor of 10 with an S-protein-bound/washing buffer (Novagen). The extract was incubated with S-protein/agarose (Novagen) at 4° C. for 12 to 18 hours. Agarose was washed with an S-protein-bound/washing buffer three times, and then with a TEV protease buffer (10 mM Tris (pH 8.0) containing 150 mM NaCl, 0.1% Nonident P-40, 0.5 mM EDTA and 1 mM DTT) once. Thereafter, the resultant was incubated with 100 units of TEV protease (Invitrogen) at 17° C. for 2 hours, so as to elute a TAP-tagged protein bound. The thus eluted protein was dialyzed against a phosphate buffer, and it was then purified with a TALON affinity purification system (Clontech). This protein complex was eluted with SDS-PAGE and SimplyBlue (Invitrogen), was then concentrated, and was then analyzed. The protein-binding product was analyzed with a MALDI/TOF mass spectrometer at the APRO Life Science Institute.

As a result of the analysis, a BRCA1-associated protein (BRAP) was identified as a protein that binds to galectin-2 (see FIG. 1a).

### Example 2

### Correlation Between Single Nucleotide Polymorphisms in BRAP Gene and Myocardial Infarction

It was revealed that galectin-2 binds to BRAP, and thus it was suggested that functional change in the BRAP gene product might be associated with the susceptibility to myocardial infarction. Thus, utilizing single nucleotide polymorphisms (SNPs) in the BRAP gene, a case-control association study was carried out on patients (3362 persons) and controls (3823 persons) according to the method described in International Publication WO2004/015100. Specifically, SNP typing was carried out by Invader assay, and the SNPs were then analyzed by a chi-square test. As a result, it was found that the number of minor homozygote (GG alleles) of the A>G SNP at nucleotide 90 in the exon 5 of the BRAP gene (registration No. rs3782886 in the NCBI SNP Database) was significantly large in myocardial infarction patients (Table 4). This result suggested that the A>G SNP at nucleotide 90 in the exon 5 of the BRAP gene was likely to be associated with myocardial infarction. In addition, it was confirmed by HapMap database (http://hapmap.org/)) that this SNP (registration No. rs3782886 in the NCBI SNP Database) has a strong linkage disequilibrium relationship with the 270 A/G SNP in the intron 3 of the same above gene (registration No. rs11066001 in the NCBI SNP Database), also with two SNPs in an aldehyde dehydrogenase 2 family (ALDH2) gene that was chromosomally adjacent to the above gene (registration Nos. rs671 and rs4646776 in the NCBI SNP Database), and also with one SNP in an FLJ30092 gene (registration No. rs2074356 in the NCBI SNP Database) Moreover, it was also found that one SNP in the ALDH2 gene (registration No. rs671 in the NCBI SNP Database) and one SNP in the FLJ30092 gene (registration No. 2074356 in the NCBI SNP Database) were associated with myocardial infarction.

### Table 4:

TABLE 4
Association of SNPs on 12q24 genomic region with myocardial infarction

| Genotype | | | | Statistics | | | | |
|---|---|---|---|---|---|---|---|---|
| AA | AG | GG | Total | Model | χ² | P | OR* | 95% CI** |

rs3782996 (BRAP)

| | | | | | | | | | | |
|---|---|---|---|---|---|---|---|---|---|---|
| MI | Number | 1378 | 1596 | 388 | 3382 | Allele | 101.1 | 8.6 × 10⁻²⁴ | 1.44 | 1.34-1.54 |
| | % | 41.0 | 47.5 | 11.5 | | Dominant | 98.2 | 3.9 × 10⁻²³ | 1.60 | 1.46-1.76 |
| Control | Number | 2014 | 1516 | 293 | 3823 | Reccessive | 31.3 | 2.2 × 10⁻⁸ | 1.57 | 1.34-1.84 |
| | % | 52.7 | 39.7 | 7.6 | | Genotype | 105.4 | 1.3 × 10⁻²³ | | |

rs671 (ALDH2)

| | | | | | | | | | | |
|---|---|---|---|---|---|---|---|---|---|---|
| MI | Number | 1447 | 1573 | 365 | 3385 | Allele | 105.3 | 1.2 × 10⁻²⁴ | 1.45 | 1.35-1.56 |
| | % | 42.7 | 48.5 | 10.8 | | Dominant | 96.1 | 1.1 × 10⁻²² | 1.59 | 1.45-1.75 |
| Control | Number | 2097 | 1507 | 259 | 3863 | Reccessive | 38.1 | 6.6 × 10⁻¹⁰ | 1.68 | 1.42-1.99 |
| | % | 54.3 | 39 | 6.7 | | Genotype | 107.6 | 4.4 × 10⁻²⁴ | | |

rs2074358 (FLJ30092)

| | | | | | | | | | | |
|---|---|---|---|---|---|---|---|---|---|---|
| MI | Number | 1587 | 1505 | 291 | 3383 | Allele | 76.2 | 4.5 × 10⁻¹⁹ | 1.4 | 1.30-1.50 |
| | % | 48.9 | 44.5 | 8.6 | | Dominant | 79.8 | 4.9 × 10⁻¹⁹ | 1.53 | 1.39-1.67 |
| Control | Number | 2196 | 1403 | 224 | 3823 | Reccessive | 20.3 | 6.5 × 10⁻⁶ | 1.51 | 1.26-1.81 |
| | % | 57.4 | 36.7 | 5.9 | | Genotype | 83.8 | 6.4 × 10⁻¹⁹ | | |

*odds ratio,
**confidence interval

### Example 3

### Confirmation of Binding of Galectin-2 to BRAP in COS7 Forced Expression System

A vector for expressing Myc-tagged galectin-2 and S-tagged BRAP in animal cells was constructed, and they were then forced to co-express in COS7 cells (an African Green Monkey kidney cell line). Thereafter, immunoprecipitation was carried out using antibodies specific to each of the expressed proteins, so as to confirm the presence or absence of the binding.

In order to carry out co-immunoprecipitation in mammalian cells, COS7 cells (Health Science Research Resources Bank; obtained from JCRB9127) were transiently transfected with Myc-tagged galectin-2 and S-tagged BRAP expression plasmids by using Fugene. The immunoprecipitation was carried out in a lysis buffer (20 mM Tris (pH 7.5) containing 150 mM NaCl and 0.2% Nonident P-40) that contained a single complete protease inhibitor tablet EDTA (Roche) per 40 ml and 5 μg ml⁻¹ MG-132 (Calbiochem). Twenty-four hours after the transfection, the transfected COS7 cells were treated with S-protein (Novagen) or Myc agarose (Santa Cruz) at 4° C. for 2 hours. Thereafter, the immunoprecipitate was washed with a lysis buffer three times. The immune mixture was visualized using horseradish peroxidase complexed with an S-protein (Novagen) or an anti-Myc peroxidase conjugate (Santa Cruz).

When immunoprecipitation was carried out using an S-protein specifically binding to the S tag of BRAP or using an anti-Myc antibody specifically binding to Myc-galectin-2, galectin-2 and BRAP were simultaneously precipitated (see FIG. 1b). This result demonstrates that galectin-2 and BRAP may directly and specifically bind to each other.

### Example 4

### Colocalization of BRAP and Galectin-2 in Vascular Smooth Muscle Cells

An antibody specific to galectin-2 was labeled with fluorescein, and an antibody specific to BRAP was labeled with rhodamine. Coronary artery vascular smooth muscle cells were immunostained. Thereafter, the expression of galectin-2 and that of BRAP were observed under a confocal scanning microscope.

Polyclonal anti-human galectin-2 and BRAP immune serums were produced in rabbits, using recombinant proteins synthesized in Escherichia coli. The polyclonal anti-human galectin-2 immune serum and BRAP were labeled with fluorescein and rhodamine, respectively, using an EZ Label™ protein labeling kit (Pierce). Human coronary artery smooth muscle cells (HCASMC; Cambrex) were cultured and were then fixed. Thereafter, the HCASMC was cultured together with the labeled antibody in a phosphate buffer saline containing 3% bovine serum albumin. The analyte was observed under an OLYMPUS FLUOVIEW confocal scanning microscope.

As a result of the observation, it was found that galectin-2 and BRAP were colocalized in the cytoplasm and nucleus (see FIG. 1c).

### Example 5

### Luciferase Assay of BRAP Intron 3 SNP in Coronary Artery Vascular Smooth Muscle Cells

A vector (BRAP promoter-luciferase) was constructed by inserting a BRAP promoter region into a pGL3 basic vector (luciferase). Thereafter, a product was constructed by inserting an oligonucleotide including an A allele or G allele of SNP once or three times into the aforementioned vector. Thereafter, vascular smooth muscle cells were transfected with the thus obtained construct, and luciferase activity was then measured.

In order to construct the BRAP promoter-pGL3 basic vector (BRAP promoter-luciferase), a DNA fragment ranging from nucleotide 21 in the 5′-side noncoding region to nucleotide-991 in the promoter region was amplified by PCR using genomic DNA as a template. The thus amplified PCR product was cloned into the KpnI and SacI restriction sites of a pGL3 basic vector (Promega) in the 5′-3′ direction. In order to construct a vector containing BRAP promoter-SNP-luciferase, a vector was constructed by cloning 1 or 3 repetitions of a double-stranded oligonucleotide (nucleotides 264-278 of intron 3) into the MluI and XhoI restriction sites of a BRAP-luciferase vector construct. HCASMC was cultured in a HCASMC growth medium (Peptide Institute, Inc.). Thereafter, using a Nucleofector System (Amaxa), the cells were transfected with 1 μg of the vector construct and 0.1 μg of a pRL-TK vector (an internal control of transfection efficiency). Twenty-four hours later, the cells were recovered, and the luciferase activity thereof was measured using a Dual-Luciferase Reporter Assay System (Promega).

As a result, stronger luciferase activity was observed in G allele than in A allele. This result demonstrates that BRAP is expressed at a higher level in an individual having G allele than in an individual having A allele (see FIG. 2a).

### Example 6

### Gel Shift Assay with BRAP Intron 3 SNP Region Sequence Oligo, Using Coronary Artery Vascular Smooth Muscle Cell Nucleic Acid Extract

According to the previously reported method, a nucleic acid extract prepared from HCASMC was incubated in the presence of 1% bovine serum albumin, together with 6 tandem copies of 16 oligonucleotides (nucleotides 264-276 in intron 3 of BRAP) labeled with digoxigenin (DIG)-11-ddUTP using a DIG gel-shift kit (Roche). The reaction was carried out at room temperature without using poly[I(dc)] reagents. As a comparison experiment, a nonlabeled oligonucleotide (125 times excessive) was pre-incubated with a nucleic acid extract before addition of the DIG-labeled oligonucleotides. A protein/DNA complex was separated using a 6% nondenaturing polyacrylamide gel (Invitrogen) in a 0.5×Tris/borate/EDTA (TBE) buffer, and it was then transferred to a nitrocellulose membrane. Thereafter, signals were detected with a chemiluminescence detection system (Roche) in accordance with the manufacturers' instructions.

As a result, the presence of an unknown factor that strongly binds to A allele could be identified (see FIG. 2b).

### Example 7

### Change in NFκB Activity by Knock-Down of BRAP and ALDH2 mRNAs in Coronary Artery Vascular Endothelial Cells

Using two types of siRNAs to BRAP and ALDH2, BRAP and ALDH2 were knocked down. Thereafter, the cells were then transfected with a vector in which luciferase bound to NFκB-specific E-selection promoter sequence (wherein NFκB activity can be indirectly measured with luciferase), and the luciferase activity thereof was then measured.

In order to knock-down the mRNAs of BRAP and ALDH2, there was used synthetic stealth (Stealth™) RNAi oligonucleotide double strand (BRAD-HSS112138 and BRAP-112139 with respect to BRAP; ALDH2-HSS100369 and ALDH2-HSS100370 with respect to ALDH2). Stealth (Stealth™) RNAi negative control high GC-containing double strand (Invitrogen) was used as a negative control. HCAEC was cultured in a HCAEC growth medium. The cells were transfected with each stealth RNAi. Twenty-four hours later, the cells were co-transfected with each stealth RNAi, pNiFty plasmid vector, and a luciferase reporter gene, to which an NFκB-specific E-selection promoter (Invivogen) had been bound by Nucleofector System (Amaxa). Twenty-four hours later, the cells were recovered, and the luciferase activity thereof was measured using a Dual-Luciferase Reporter Assay System. The mRNA level was quantified using SYBR Green and ABI 7700 Sequence Detection System.

The NFκB activity was decreased by the knock-down of BRAP. In contrast, by the knock-down of ALDH2, the NFκB activity was increased (see FIG. 2c).

### INDUSTRIAL APPLICABILITY

Utilizing the SNPs identified by the present invention, it becomes possible to provide a method for diagnosing inflammatory diseases such as myocardial infarction or a method for developing a therapeutic agent for inflammatory diseases.

### BRIEF DESCRIPTION OF THE DRAWINGS

FIG. 1 shows: (a) identification of BRAP according to a TAP method; (b) confirmation of the binding of BRAP to galectin-2 in a COST forced expression system; and (c) colocalization of galectin-2 and BRAP in vascular smooth muscle cells.

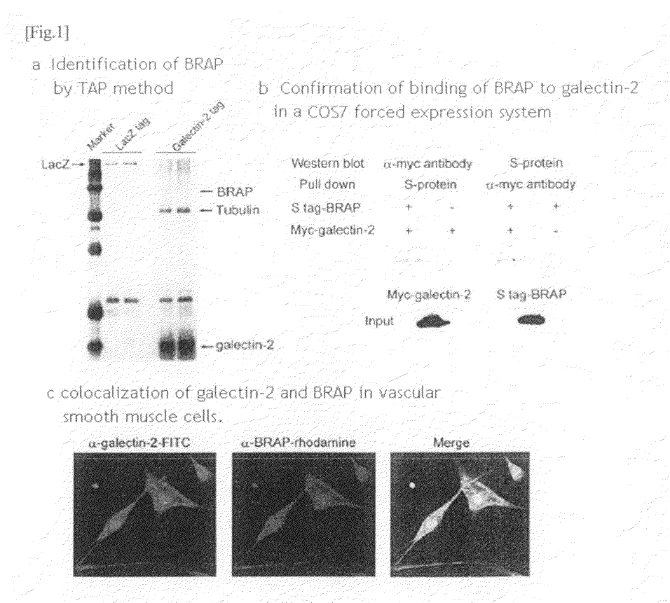

FIG. 2 shows: (a) the luciferase assay of BRAP intron 3 A/G SNP in coronary artery vascular smooth muscle cells; (b) the gel shift assay of a BRAP intron 3 A/G SNP region using a coronary artery vascular smooth muscle cell extract solution; and a change in NFκB activity by the knock-down of BRAP and ALDH2 in coronary artery vascular endothelial cells.

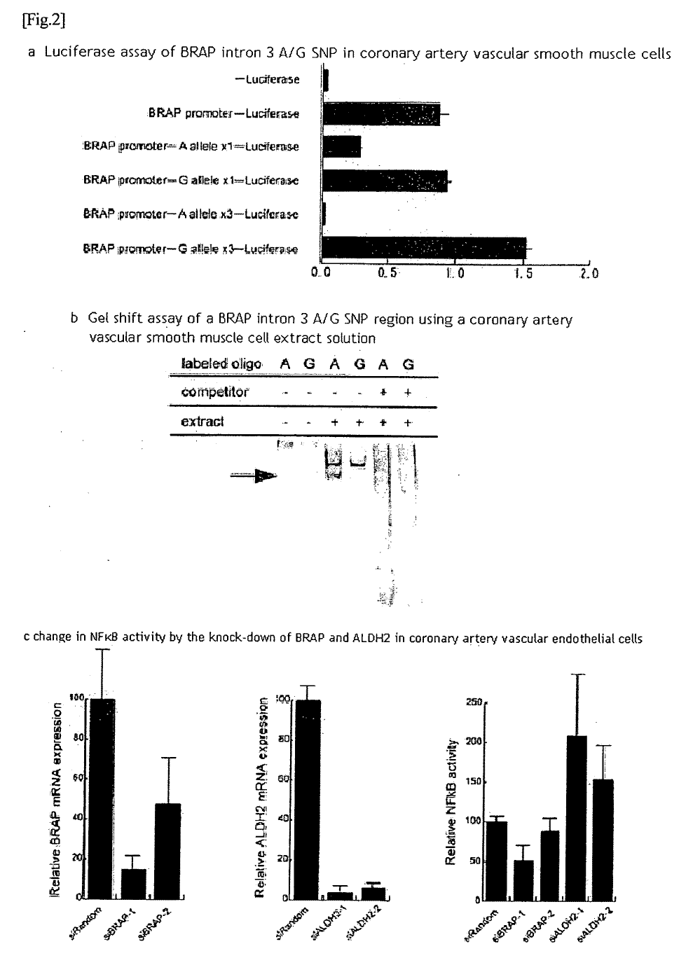

1. A method for judging inflammatory diseases, which comprises detecting at least one gene polymorphism in the BRCA1-associated protein (BRAP) gene.

2. A method for judging inflammatory diseases, which comprises detecting at least one single nucleotide polymorphism in the BRCA1-associated protein (BRAP) gene.

3. A method for judging inflammatory diseases, which comprising detecting any one of the following polymorphisms:
(i) the A/G polymorphism at nucleotide 90 in the nucleotide sequence of exon 5 of the BRCA1-associated protein (BRAP) gene as shown in SEQ ID NO: 1 (registration No. rs3782886 in the NCBI SNP Database);
(ii) the A/G polymorphism at nucleotide 270 in the nucleotide sequence of intron 3 of the BRCA1-associated protein (BRAP) gene as shown in SEQ ID NO: 2 (registration No. rs110660001 in the NCBI SNP Database); and
(iii) a polymorphism that is in a linkage disequilibrium state in which an r-square value used as a linkage disequilibrium index is 0.8 or greater with respect to the polymorphism described in (i) or (ii) above.

4. The method according to claim 1, wherein the inflammatory disease is myocardial infarction.

5. An oligonucleotide, which can hybridize to a sequence consisting of at least 10 continuous nucleotides including the A/G polymorphism at nucleotide 90 in the nucleotide sequence of exon 5 of the BRCA1-associated protein (BRAP) gene as shown in SEQ ID NO: 1 or at nucleotide 270 in the nucleotide sequence of intron 3 of the BRCA1-associated protein (BRAP) gene as shown in SEQ ID NO: 2, or a complementary sequence thereof, and which is used as a probe in the method according to claim 1.

6. An oligonucleotide, which can amplify a sequence consisting of at least 10 continuous nucleotides including the A/G polymorphism at nucleotide 90 in the nucleotide sequence of exon 5 of the BRCA1-associated protein (BRAP) gene as shown in SEQ ID NO: 1 or at nucleotide 270 in the nucleotide sequence of intron 3 of the BRCA1-associated protein (BRAP) gene as shown in SEQ ID NO: 2, and/or a complementary sequence thereof, and which is used as a primer in the method according to 1.

7. The oligonucleotide according to claim 6, wherein the primer is a forward primer and/or a reverse primer.

8. A kit for diagnosing inflammatory diseases, which comprises at least one oligonucleotide according to claim 5.

9. The kit according to claim 8, wherein the inflammatory disease is myocardial infarction.

10. A method for analyzing the expression state of a BRCA1-associated protein (BRAP), which comprises detecting the A/G polymorphism at nucleotide 90 in the nucleotide sequence of exon 5 of the BRCA1-associated protein (BRAP) gene as shown in SEQ ID NO: 1 or the A/G polymorphism at nucleotide 270 in the nucleotide sequence of intron 3 of the BRCA1-associated protein (BRAP) gene as shown in SEQ ID NO: 2.

11. A method for screening for a therapeutic agent for inflammatory diseases, which comprises steps of analyzing the expression level of a BRCA1-associated protein (BRAP) or the function of a BRCA1-associated protein (BRAP) in cells in the presence of a candidate substance, and selecting a substance that suppresses said expression level or a substance that inhibits or modifies said function.

12. A method for measuring the transcriptional activity of a BRCA1-associated protein (BRAP), which comprises introducing a BRAP gene fragment containing the A/G polymorphism at nucleotide 270 in the nucleotide sequence of intron 3 of the BRCA1-associated protein (BRAP) gene as shown in SEQ ID NO: 2 into cells, culturing the cells, and analyzing the expression of said gene.

13. A method for screening for a substance that inhibits or promotes the transcriptional activity of a BRCA1-associated protein (BRAP), which comprises introducing a BRAP gene fragment containing the A/G polymorphism at nucleotide 270 in the nucleotide sequence of intron 3 of the BRCA1-associated protein (BRAP) gene as shown in SEQ ID NO: 2 into cells, culturing the cells in the presence of a candidate substance that inhibits or promotes the transcriptional activity of BRAP, and analyzing the expression of said gene.

14. A method for screening for a transcription-regulatory factor of a BRCA1-associated protein (BRAP), which comprises bringing a BRAP gene fragment containing the A/G polymorphism at nucleotide 270 in the nucleotide sequence of intron 3 of the BRCA1-associated protein (BRAP) gene as shown in SEQ ID NO: 2 into contact with a sample assumed to comprise the transcription-regulatory factor of BRAP, and detecting the binding between the aforementioned gene fragment and the transcription-regulatory factor.


SEQUENCE LISTING (2 sequences)

SEQ ID NO: 1 - DNA, 114 bp, Homo sapiens
gctgatgcggatagtttttatatgacatgcaatggccgccagttcaactcaatagaagatgacgtttgccagctagtgtatgtggaaagrgctgaagtgctcaaatctgaagat

SEQ ID NO: 2 - DNA, 2296 bp, Homo sapiens
gtaagaaaaccctcttttcttgggggttagtcaatttttttttttttttttttgctttgctttagcatagtctcttgtcctgaaggaaagtgggtattttaagcttcaaaataataatacttttgtctgttgtatgttagagaattctcagatgttttacatttgattaatcttgtggattgggtcaaagcttttccttataaaagaaatgcttgtggatacacttaagagaatatgtgggaaaaatgttttctttttttttttttgaartggagtttcggtctcgttgcccaggctggagtgcaatggcgcgatctcagatcactgcatcttctgcctcctgagttcaagtgattctccttcctcagcctcccaagtagctgggattacaggtgcccaccaccacacctagctaattttttgtatttttagtagagatggggtttcaccctgttggccaggctggtcttgaactccgacctcaggttatccgcccgcctcagcctcccaaagtgctgggattacaggcgtgagccactgcgccctgccaggaaaatgttttcttagcagataggcatggaaggtgatggggaattgaaaggacaaagaaaatgatccacagcatctttgtagtctcatgtccccaccaaatctttactgaagaggctgctaagaagtcagttaactcattacaattttattacctcttttttaccattttctgtggttcaaacaaagcttgctagagccccaaggaaaggaagcatttggctagataatccacaagcccagccactaccttcttgggtaatggtagctgataccttaaggctgttcttgtgctaactcatttgccagatctctactgggaaaacagtgactattcaggtagccattttgttttttgttttgttttttgttttgttttttgttttgtttttttttgagacagggtctcactccataactcaggctggagtgcagtggtgcaatcctggctcactgcaacctcccctcccgggttcaagcaattctcctgcctcagagtagctgggaccacaggtgcacaccaccacgcccagttaattttttttttttttttggcagagatggggtttcatcatgttggctaggctggtctcgaactcctaacctcaagtgatcagcccacctcagcctcccaaagtgttgggattacaggtgtgagtcactgcacccggcacaggtagccattctggatcacagctgtcaagtgagtttgcagcatttcaaaaaacaaattgcattgtgggaggattttctttcctctctattccagccatctcaacttggtaacacttcctgtaatcctcagttacatcctcatggtttagaattctctcttgccgggcgtggtggctcacgcctgtaatcccagtactttgggaggccgaggcgggggatcacgaggtcaggagttcgaaaccagcctggccaacatagtgaaaccccatctctactaaaaatacaaaaattagcccggcgtggtggcacgcgtctgtaatcccagctacttgggaggctgagcaggagaattgcttgaacccaggagtcagaggttgcagtgagccgagaccatgcagcaagactctgaaaaaaaaaaagacagaattctctcgtatatttagagctgtttttttaggccttttaagagaggtccatagccagagttgtgcttttaagttaaccagtgacagatttttttttttttttttttttttgagatggagtcttgctctgtggcccaggctggagtgcaatggcgcgatattggctcactgcaagctctgcctcccaggttcacaccattctcctgcctcagcctcccaagtagctgggactacaagcacctgccaccatgcccggctaattttttttgtatttttagtagagatggggtttcaccgtgttagccaggatggtctcgatctcctgacctcgtgatccacccgcctcggcctgccaaagtgctgggattacgagcatgagctactgtgcctggccaccagtgacagattattattcaaacattgatgagagtaaaacactgcaaatttaccgaatgcagatttataaccgtaataaatgttgaactgaagattaaaaagtccaatggggggtataatatagaacagttcattaatgaaatattacaatttttattttttcctgttttcttggacataaaagttgtctagtattgtttaaactaaccatacctcatctattcaagacactctaattttttttttgttag